(19) United States
(12) Patent Application Publication
(10) Pub. No.: US 2021/0000350 A1
(43) Pub. Date: Jan. 7, 2021

(54) NEUROLOGICAL EXAMINATION SYSTEM

(71) Applicant: KONINKLIJKE PHILIPS N.V., EINDHOVEN (NL)

(72) Inventors: LYUBOMIR GEORGIEV ZAGORCHEV, BURLINGTON, MA (US); JOEL HAAF, SAN DIEGO, CA (US)

(21) Appl. No.: 16/982,720

(22) PCT Filed: Mar. 20, 2019
(86) PCT No.: PCT/EP2019/056900

§ 371 Date: Sep. 21, 2020

Related U.S. Application Data
(60) Provisional application No. 62/645,931, filed on Mar. 21, 2018.

(51) Int. Cl. A61B 5/00; A61B 5/055; A61B 5/0476; A61B 5/04; G01R 33/56; G06T 7/00

(52) U.S. Cl. CPC A61B 5/0042; A61B 5/055; A61B 5/7425; A61B 5/7275; A61B 5/0476; G06T 2207/30016; G01R 33/5608; G06T 7/0014; A61B 2576/026; G06T 2207/10088; A61B 5/04008

(57) ABSTRACT

Systems and methods for evaluating an anatomical structure in a brain of a subject are provided. In an embodiment, a system for evaluating an anatomical structure in a brain of a subject includes a computing device in communication with a magnetic resonance imaging (MRI) device. The computing device operable to determine an abnormality in the anatomical structure by comparing a test activation level within a geometry of the anatomical structure to data in a normative database, and output, to a display device, a graphical representation of the abnormality in the anatomical structure. The test activation level is determined by aligning functional magnetic resonance imaging (fMRI) data obtained by use of the MRI device and the geometry of the anatomical structure. The geometry of the anatomical structure is delineated based on segmentation of magnetic resonance (MR) data obtained by use of the MRI device. The data in the normative database include activation levels of the anatomical structure of a plurality of neurologically non-diseased subjects.

### TECHNICAL FIELD

The present disclosure relates generally to neurological examination, and in particular, to neurological examination systems and methods for identifying an abnormality in a subject's brain and determining a probability of a neurological disorder.

### BACKGROUND

Due to its high spatial resolution and excellent soft tissue contrast, structural magnetic resonance imaging (MRI) is well suited for detection of cerebral and sub-cortical atrophy as well as longitudinal tracking of white/grey matter changes. MRI has different variations and modalities that are useful in diagnosing neurological disorders.

Functional magnetic resonance imaging (fMRI) is a variation of MRI that utilizes the magnetic properties of oxygenated and deoxygenated hemoglobin that result in different signal intensity values. Temporal changes in the ratio of oxygenated to deoxygenated blood are used to generate images of task-related metabolic activity. Tasks that increase regional blood brain activity, and ultimately regional oxygen demand and blood flow, may be performed by a subject in an MRI scanner and used to study cognitive function in individuals with neurological disease and mental disorders.

Diffusion tensor imaging (DTI) is another MRI modality that utilizes the properties of water diffusion to provide information about connectivity and functional integrity of brain tissues and underlined white matter tracts. DTI is based on the principle(s) that water molecules diffuse along the principal axes of tensors describing the local rate of diffusion. The tensors are centered at voxels in three dimensions and can be visualized as ellipsoids. As a result, voxels along white matter tracts form diffusion lines, also known as fiber tracts, if viewed along the long axis of their individual tensors. DTI tractography is an image processing technique that traces such ellipsoids along their long axis by starting from a user defined seed point/region.

Electroencephalography (EEG) and magnetoencephalography (MEG) can be used, for example, to study neurological disorders such as Alzheimer's disease, epilepsy, traumatic brain injury, and epilepsy. Both EEG and MEG measure ionic current within neurons of the brain. The ionic current within neurons can be referred to as neuronal current. EEG measures voltage fluctuations resulting from the neuronal current, while MEG measures the magnetic field induced by the neuronal current. By measuring the neuronal current, both EEG and MEG can be used to evaluate brain activity. MEG data and EEG data can therefore supplement fMRI data as they measure different aspects of brain activities. MEG data and EEG data can also be cross-compared to DTI data as fiber tracts in DTI data are depictions of neuronal connectivity in a subject's brain.

Conventionally, fMRI data, DTI data, EEG data, and MEG data are usually analyzed separately by region. Tracking and quantitative analysis of these data on an anatomical-structure-by-anatomical-structure basis are not available. For the same reasons, normative data and biomarkers are not created and developed on an anatomical-structure-by-anatomical-structure basis either. Therefore, there is a need for an improved neurological examination system and method.

### SUMMARY

Embodiments of the present disclosure are configured to identify an abnormality in a subjectsubject's brain by comparing the anatomical-specific fMRI, DTI, EEG and MEG data to data in a normative database and to determine a probability of a neurological disorder by comparing the anatomical-specific fMRI, DTI, EEG and MEG data to data in a biomarker database. The data in the normative database includes anatomical-specific fMRI, DTI, EEG and MEG data of healthy subjects who have not been diagnosed with a neurological disorder, as well as non-imaging data such as genomics, electronic medical records, radiology reports of these healthy subjects. The data in the biomarker database includes anatomical-specific fMRI, DTI, EEG and MEG data of subjects who have been diagnosed with having been diagnosed with a neurological disorder, as well as non-imaging data such as genomics, electronic medical records, radiology reports of these subjects with neurological disorders.

Systems and methods for evaluating an anatomical structure in a brain of a subject are provided. In an embodiment, a system for evaluating an anatomical structure in a brain of a subject includes a computing device in communication with a magnetic resonance imaging (MRI) device. The computing device operable to determine an abnormality in the anatomical structure by comparing a test activation level within a geometry of the anatomical structure to data in a normative database, and output, to a display device, a graphical representation of the abnormality in the anatomical structure. The test activation level is determined by aligning functional magnetic resonance imaging (fMRI) data obtained by use of the MRI device and the geometry of the anatomical structure. The geometry of the anatomical structure is delineated based on segmentation of magnetic resonance (MR) data obtained by use of the MRI device. The data in the normative database include activation levels of the anatomical structure of a plurality of neurologically non-diseased subjects.

In some embodiments, the computing device is further operable to determine a probability of a neurological disorder by comparing the test activation level associated with the abnormality to data in a biomarker database. The data in the biomarker database include activation levels of the anatomical structure of a plurality of neurologically diseased subjects. The graphical representation includes the probability of the neurological disorder. In some embodiments, the computing device is further operable to determine the abnormality in the anatomical structure by comparing a test electrical activity level within the geometry of the anatomical structure to the data in the normative database, and determine the probability of the neurological disorder by comparing the test electrical activity level associated with the abnormality to the data in the biomarker database. The test electrical activity level is determined by aligning electroencephalography (EEG) data obtained by use of an EEG device and the geometry of the anatomical structure. The computing device is in communication with the EEG device. The data in the normative database include electrical activity levels of the anatomical structure of the plurality of neurologically non-diseased subjects. The data in the biomarker database include electrical activity levels of the anatomical structure of the plurality of neurologically diseased subjects.

In some embodiments, the computing device is further operable to determine the abnormality in the anatomical structure by comparing a test neuronal activity level within the geometry of the anatomical structure to the data in the normative database, and determine the probability of the neurological disorder by comparing the test neuronal activity level associated with the abnormality to the data in the biomarker database. The test neuronal activity level is determined by aligning magnetoencephalography (MEG) data obtained by use of an MEG device and the geometry of the anatomical structure. The computing device is in communication with the MEG device. The data in the normative database include neuronal activity levels of the anatomical structure of the plurality of neurologically non-diseased subjects. The data in the biomarker database include neuronal activity levels of the anatomical structure of the plurality of neurologically diseased subjects.

In some embodiments, the computing device is further operable to determine the abnormality in the anatomical structure by comparing a test fiber tract density within the geometry of the anatomical structure to the data in the normative database, and determine the probability of the neurological disorder by comparing the test fiber tract density associated with the abnormality to the data in the biomarker database. The test fiber tract density is determined by aligning diffusion tensor imaging (DTI) data obtained by use of the MRI device and the geometry of the anatomical structure. The data in the normative database include fiber tract densities of the anatomical structure of the plurality of neurologically non-diseased subjects. The data in the biomarker database include fiber tract densities of the anatomical structure of the plurality of neurologically diseased subjects. In some implementations, the graphical representation includes a treatment recommendation. In some implementations, the graphical representation includes a prescription recommendation. In some embodiments, the graphical representation includes a report. In some embodiments, the system further includes the MRI device and the display device.

In another embodiment, a system for evaluating an anatomical structure in a brain of a subject includes a computing device in communication with a magnetic resonance imaging (MRI) device. The computing device is operable to determine a probability of the neurological disorder by comparing a test activation level within a geometry of the anatomical structure to data in a biomarker database, and output, to a display device, a graphical representation of the probability of the neurological disorder. The test activation level is determined by aligning functional magnetic resonance imaging (fMRI) data obtained by use of the MRI device and the geometry of the anatomical structure. The geometry of the anatomical structure is delineated based on segmentation of magnetic resonance (MR) data obtained by use of the MRI device. The data in the biomarker database include activation levels of the anatomical structure of a plurality of neurologically diseased subjects.

In some embodiments, the computing device is further operable to determine an abnormality in the anatomical structure by comparing a test activation level within the geometry of the anatomical structure to data in a normative database. The data in the normative database include activation levels of the anatomical structure of a plurality of neurologically non-diseased subjects. The graphical representation includes the abnormality in the anatomical structure. In some embodiments, the computing device is further operable to determine the abnormality in the anatomical structure by comparing a test electrical activity level within the geometry of the anatomical structure to the data in the normative database, and determine the probability of the neurological disorder by comparing the test electrical activity level to the data in the biomarker database. The test electrical activity level is determined by aligning electroencephalography (EEG) data obtained by use of an EEG device and the geometry of the anatomical structure. The computing device is in communication with the EEG device. The data in the normative database include electrical activity levels of the anatomical structure of the plurality of neurologically non-diseased subjects. The data in the biomarker database include electrical activity levels of the anatomical structure of the plurality of neurologically diseased subjects.

In some embodiments, the computing device is further operable to determine the abnormality in the anatomical structure by comparing a test neuronal activity level within the geometry of the anatomical structure to the data in the normative database, and determine the probability of the neurological disorder by comparing the test neuronal activity level to the data in the biomarker database. The test neuronal activity level is determined by aligning magnetoencephalography (MEG) data obtained by use of an MEG device and the geometry of the anatomical structure. The computing device is in communication with the MEG device. The data in the normative database include neuronal activity levels of the anatomical structure of the plurality of neurologically non-diseased subjects. The data in the biomarker database include neuronal activity levels of the anatomical structure of the plurality of neurologically diseased subjects.

In some implementations, the computing device is further operable to determine the abnormality in the anatomical structure by comparing a test fiber tract density within the geometry of the anatomical structure to the data in the normative database, and determine the probability of the neurological disorder by comparing the test fiber tract density to the data in the biomarker database. The test fiber tract density is determined by aligning diffusion tensor imaging (DTI) data obtained by use of the MRI device and the geometry of the anatomical structure. The data in the normative database include fiber tract densities of the anatomical structure of the plurality of neurologically non-diseased subjects. The data in the biomarker database include fiber tract densities of the anatomical structure of the plurality of neurologically diseased subjects. In some implementations, the graphical representation includes a treatment recommendation. In some instances, the graphical representation includes a prescription recommendation. In some embodiments, the graphical representation includes a report. In some embodiments, the system includes the MRI device and the display device.

Other devices, systems, and methods specifically configured to interface with such devices and/or implement such methods are also provided.

Additional aspects, features, and advantages of the present disclosure will become apparent from the following detailed description along with the drawings.

### BRIEF DESCRIPTIONS OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is a schematic diagram of a neurological examination system, according to aspects of the present disclosure.

FIG. 2 is a flowchart illustrating a method of building a normative database and a biomarker database for anatomical-structure-specific analysis, according to aspects of the present disclosure.

FIG. 3 is a flowchart illustrating a method of for determining an abnormality in an anatomical structure in a brain of a subject and a probability of a neurological disorder, according to aspects of the present disclosure.

FIG. 4 is a schematic diagram illustrating a process flow for segmenting MR image to delineate a geometry of an anatomical structure, according to aspects of the present disclosure. FIG. 4 (410), FIG. 4 (420), FIG. 4 (430), FIG. 4 (440), FIG. 4 (450), and FIG. 4 (460) illustrate black and white versions of items shown in FIG. 4.

FIG. 5 is a graphical representation of activation levels within an anatomical structure of the brain, according to aspects of the present disclosure.

FIG. 6 is an MR image of a subject's brain overlaid with a segmented model of the subject's amygdala-hippocampal complex (AHC), according to aspects of the present disclosure.

FIG. 7 is an MR image of a subject's brain overlaid with fiber tracts passing through the segmented model of the subject's AHC, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates.

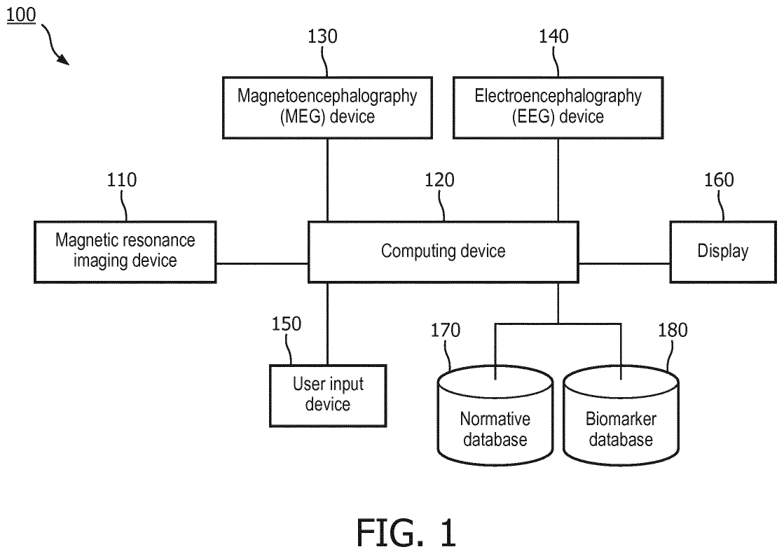

Referring now to FIG. 1, shown therein is a schematic diagram of a neurological examination system 100 according to some embodiments of the present disclosure. The system 100 includes a computing device 120 in electrical communication with a magnetic resonance imaging (MRI) device 110, a magnetoencephalography (MEG) device 130, an electroencephalography (EEG) device 140, a user input device 150, and a display 160. The computing device 120 includes a processing circuit, such as one or more processors in communication with memory. The memory can be tangible computer readable storage media that stores instructions that are executable by the one or more processors. In some embodiments, the computing device 120 can be a workstation or a controller that serves as an interface between the MRI device 110, the MEG device 130, the EEG device 140, on the one hand, and the display 160, on the other hand. In some other embodiments, the computing device 120 only controls the MRI device 110. In those embodiments, the computing device 120 can access data obtained by use of the MEG device 130 and the EEG device 140 but do not directly control their operation. In some embodiments, the MRI device 110 can operate in different modalities, including but not limited to magnetic resonance (MR) imaging, diffusion tensor imaging (DTI) and functional magnetic resonance imaging (fMRI) and output imaging data to the computing device 120. In some implementations, the MRI device 110 can operate in different modalities at the same time. For example, the MRI device can perform MR scans and DTI scans simultaneously.

In some embodiments, the computing device 120 can receive MR data from the MRI device 110, process the same and output MR image data to the display 160 such that the display 160 can display MR images. In some embodiments, the computing device 120 can receive fMRI data from the MRI device 110, process the same and output the fMRI data to the display 160. In some embodiments, the computing device 120 can align or co-register the MR data and the fMRI data through suitable processes, such as survey scans, rigid registration, volume localization and direction cosines. In some embodiments, the acquisition of fMRI data does not conclude until a predetermined or threshold activation levels are attained. In some embodiments, the computing device 120 can receive data from the MEG 130, process the same to determine neuronal activity levels and output the neuronal activity levels to the display 160. Similarly, in some embodiments, the computing device 120 can receive data from the EEG 140, process the same to determine electrical activity levels and output the electrical activity levels to the display 160. In some embodiments, the computing device 120 can align or co-register the MR data and the EEG and MEG data through suitable processes, such as survey scans, rigid registration, volume localization and direction cosines. In some implementations, the EEG device 140 is compatible with MRI device 110 and EEG data can be obtained simultaneously with the MR scan. In those implementations, the MR data and the EEG data should be aligned or co-registered under the same field of view. In some embodiments, the computing device 120 can receive DTI data from the MRI device 110, process the same to identify fiber tracts and output the identified fiber tracts to the display 160. In those embodiments, the MR data and the DTI data can be obtained simultaneously or in sequence with the MR scan. If the subject being scanned remains still during the MR/DTI scan, a survey scan should be sufficient to align the field of view of the MR scan and that of the DTI scan. If the subject moves, additional survey scans may need to be performed to ensure appropriate alignment between the MR scan and the DTI scan. In some instances, the computing device 120 can align or co-register the MR data and the DTI data through suitable processes, such as rigid registration, volume localization and direction cosines.

In some embodiments, the MR data can be T1 weighted (T1W) MR images and the computing device 120 can automatically segment the MR image to delineate geometries of anatomical structures in the brain of the subject. In some implementations, the computing device 120 can segments the MR image data based on a three-dimensional (3D) brain model. In some instances, the 3D brain model is received by the computing device 120 from a storage media or through wired or wireless connection to a server or a remote workstation. In some other instances, the 3D brain model can be stored in a storage device in the computing device 120 or a storage device retrievable by the computing device 120. In some implementations, the 3D brain model is a shape-constrained deformable brain model. In some instances, the 3D brain model may be the brain model described in “Evaluation of traumatic brain injury subjects using a shape-constrained deformable model,” by L. Zagorchev, C. Meyer, T. Stehle, R. Kneser, S. Young and J. Weese, 2011, in Multimodal Brain Image Analysis by Liu. T., Shen D., Ibanez L., Tao X. (eds). MBIA 2011. Lecture Notes in Computer Science, vol. 7012. Springer, Berlin, Heidelberg, the entirety of which is hereby incorporated by reference. In some embodiments, the 3D brain model may be the deformable brain model described in U.S. Pat. No. 9,256,951, titled “SYSTEM FOR” PID AND ACCURATE QUANTITATIVE ASSESSMENT OF TRAUMATIC BRAIN INJURY″ or the shape-constrained deformable brain model described in U.S. Pat. App. Pub. No. 20150146951, titled “METHOD AND SYSTEM FOR QUANTITATIVE EVALUATION OF IMAGE SEGMENTATION,” each of which is hereby incorporated by reference in its entirety.

In some embodiments, the automatic segmentation not only delineates the geometries of anatomical structures in the brain but also defines a plurality of voxels in each of the geometries. With the MR data aligned with the fMRI data, DTI data, the EEG data and the MEG data, the geometries and voxels can be transferred to the fMRI, DTI, EEG, MEG space or the fMRI image, DTI image, EEG image, and MEG image can be overlaid on the MR image. In some implementations, based on the fMRI data from the MRI device 110, the computing device 120 can determine an activation level within a voxel, wherein the activation level can be an accumulated activation level, an instantaneous activation level, a time-average activation level, or an event-average activation level. With the activation level for each of the voxel known, the computing device 120 can then determine an activation level within a geometry of an anatomical structure by integrating the activation levels of all voxels within the geometry. In some embodiments, the computing device 120 can use color coding to denote different activation levels, be they accumulated activation levels, instantaneous activation levels, time-average activation levels, or event-average activation levels. In some implementations, the computing device 120 can also output activation level contours within a geometry based on the activation level of the voxels in the geometry. In some embodiments, the computing device 120 can output a graphical representation of the determined activation levels within the geometry to the display 160.

In some embodiments, the MR data and fMRI data include information about multiple geometries of different anatomical structures of the subject's brain. When tasks designed to increase regional brain activity are administrated to the subject, the activation levels within geometries of different anatomical structures may assume a sequence or pattern over time. For example, a first high average activation level can be observed within a first anatomical structure, and then a second high average activation level can be observed within a second anatomical structure. The computing device 120 can also determine a sequence or pattern of activation among the anatomical structures.

In some implementations, based on the DTI data from the MRI device 110, the computing device 120 can identify fiber tracts passing through a geometry of an anatomical structure. In some embodiments, the computing device 120 can determine the fiber tract density within the geometry of the anatomical structure. In some implementations, the fiber tract density includes a ratio of fiber tract volume out of the total volume of the geometry of the anatomical structure. In some implementations, the computing device 120 can use color coding to denote different fiber tract densities in different anatomical structures. In some embodiments, the computing device 120 can output a graphical representation of the fiber tract density within the geometry to the display 160. In some implementations, based on the MEG data from the MEG device 130, the computing device 120 can identify neuronal activity level within a geometry of an anatomical structure. In some implementations, the computing device 120 can use color coding to denote different levels of neuronal activity levels in different anatomical structures. In some embodiments, the computing device 120 can output a graphical representation of the neuronal activity level within the geometry to the display 160. In some implementations, based on the EEG data from the EEG device 140, the computing device 120 can identify electrical activity level within a geometry of an anatomical structure. In some implementations, the computing device 120 can use color coding to denote different levels of electrical activity levels in different anatomical structures. In some embodiments, the computing device 120 can output a graphical representation of the electrical activity level within the geometry to the display 160.

In some embodiments, the computing device 120 can be used to build a normative database and a biomarker database. In those embodiments, the computing device 120 can receive a diagnosis the subject. If the diagnosis is negative and indicative of a healthy brain, then the subject is identified as a neurologically non-diseased subject and the fMRI activation levels, sequence of activation, DTI fiber tract densities, MEG neuronal activity levels, EEG electrical activity levels within respective anatomical structures of the subject can be stored by the computing device 120 in a normative database 170 in communication with the computing device 120. If, however, the diagnosis is positive and indicative of a neurological disorder, then the subject is identified as a neurologically diseased subject and the aforementioned anatomical-structure-specific data can be stored in a biomarker database 180. Over time, the normative database can include fMRI activation levels, sequence of activation, DTI fiber tract densities, MEG neuronal activity levels, EEG electrical activity levels within respective anatomical structures of a plurality of neurologically non-diseased subjects and the biomarker database can include fMRI activation levels, sequence of activation, DTI fiber tract densities, MEG neuronal activity levels, EEG electrical activity levels within respective anatomical structures of a plurality of neurologically diseased subjects. In some embodiments, the computing device 120 can normalize the data in the normative database 170 and the biomarker database 180 based on head sizes or head shape descriptors of the pluralities of neurologically non-diseased or diseased subjects. That way, the variations due to head size can be taken into account to provide more accurate dataset for comparison.

In some embodiments, the computing device 120 can associate with the diagnosed neurological disorder, the fMRI activation levels, sequence of activation, DTI fiber tract densities, MEG neuronal activity levels, and EEG electrical activity levels with the diagnosis. Because the data stored in the biomarker database 180 are all normalized with respect to each anatomical structure, the activity levels, neuronal activity levels, electrical activity levels, and DTI fiber densities can be meaningfully quantified and analyzed with respect to the diagnosed neurological disorder. The same cannot be said for conventional use of the same data. For example, the conventional DTI identifies fiber tracts within a subject's brain. However, without a meaningfully defined space or geometry, the fiber tract density and the ratio of fiber tract volume can neither be calculated nor cross-compared to corresponding values from a different subject. In some embodiments, the computing device 120 can statistically identify characteristics or biomarkers in terms of activation levels, sequences of activation, neuronal activity levels, electrical activity, and fiber tract density.

In some embodiments, after the computing device 120 receives fMRI data and DTI data of a subject from the MRI device 110, MEG data of the subject from the MEG device 130, EEG data of the subject from the EEG device 140, the computing device 120 can compare the subject's activation level, sequence of activation, neuronal activity level, electrical activity level, and fiber tract density to the data stored in the normative database 170 on an anatomical-structure-by-anatomical-structure basis. Such comparison allows the computing device 120 to identify an abnormality with respect to a specific anatomical structure. In some embodiments, once an abnormality is identified, the computing device 120 can compare the subject's activation level, sequence of activation, neuronal activity level, electrical activity, and fiber tract density within the anatomical structure to the data stored in the biomarker database 180. The computing device 120 can determine if the activation level, sequence of activation, neuronal activity level, electrical activity level, and fiber tract density of the “abnormal” anatomical structure matches a characteristic pattern or biomarker of a neurological disorder and how probable is the abnormality indicative of the neurological disorder. In an alternative arrangement, the computing device 120 can access both the normative database 170 and the biomarker database 180 in parallel in identifying the abnormality and determining the probability of the neurological disorder. In some embodiments, whenever the diagnosis is positive, any information on a treatment recommendation for recommended therapy or procedures, and a prescription recommendation, for recommended medication are also stored in the biomarker database 180. In those embodiments, besides a probability of a neurological disorder, the computing device 120 can also determine a treatment recommendation and a prescription recommendation based on the recommended treatments and prescriptions in the biomarker database 180. The anatomical-structure-specific nature of the activation level, sequence of activation, neuronal activity level, electrical activity level, and fiber tract density obtained according to the present disclosure allows for in-exam tracking of changes in brain activities and longitudinal tracking across different examinations.

In some implementations, the computing device 120 can generate and output to the display 160 a graphical representation of the identified abnormality, the probability of the neurological disorder, the recommended treatment, and the recommended prescriptions. The graphical representation can include color contours, text, pop-up dialog boxes, clickable hyperlinks. In some implementation, the graphical representation can assume a form of a report.

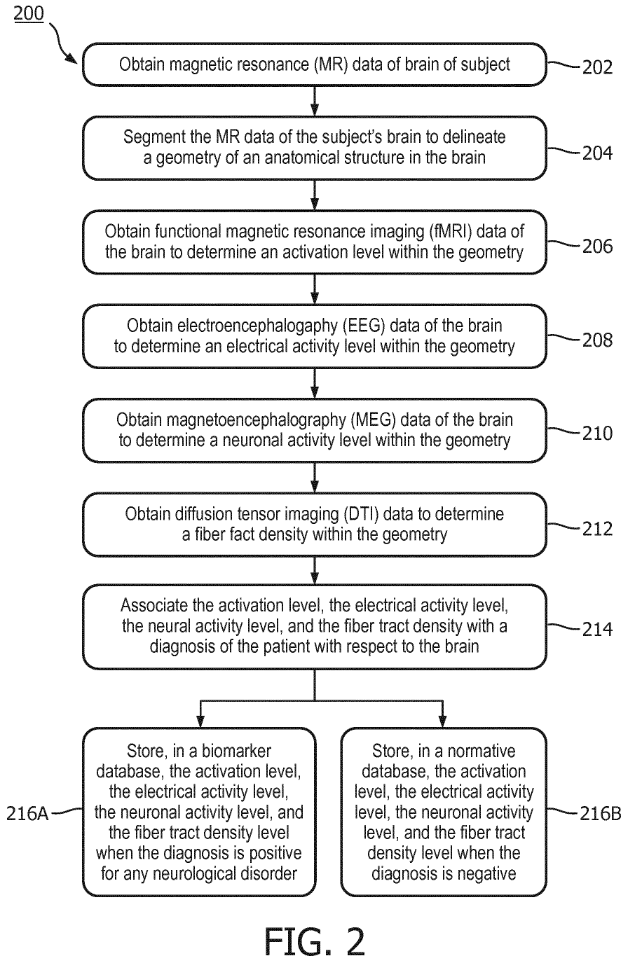

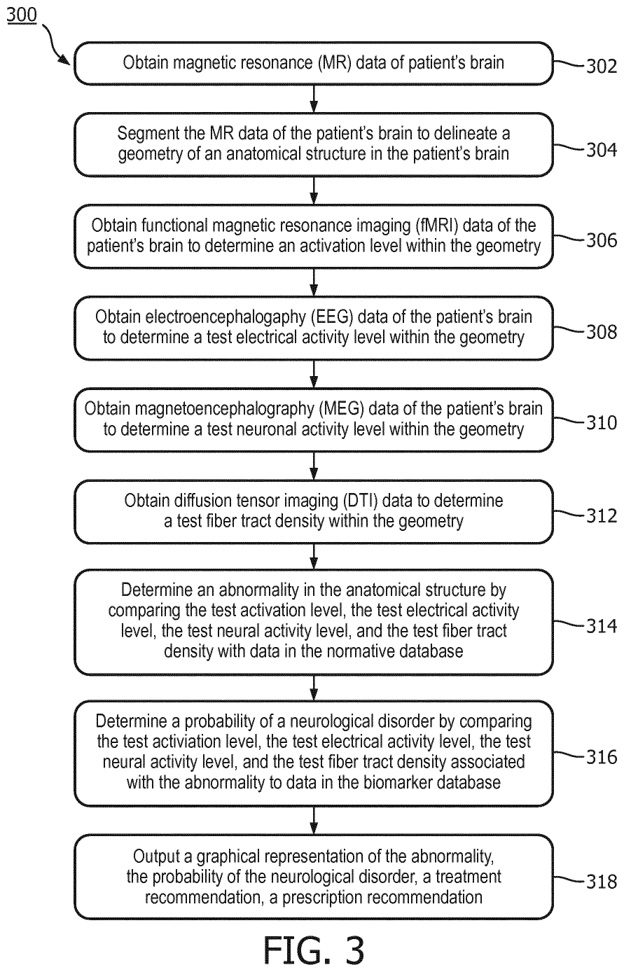

Referring now to FIG. 2, shown therein is a flowchart illustrating am exemplary method 200 of building a normative database and a biomarker database for anatomical-structure-specific analysis. The method 200 includes operations 202, 204, 206, 208, 210, 212, 214, 216A, and 216B. It is understood that the operations of method 200 may be performed in a different order than shown in FIG. 2, additional operations can be provided before, during, and after the operations, and/or some of the operations described can be replaced or eliminated in other embodiments. The operations of the method 200 can be carried out by a computing device in the MRI system, such as the computing device 120 of the system 100. The method 200 will be described below with reference to FIGS. 3, 4, 5, 6, and 7.

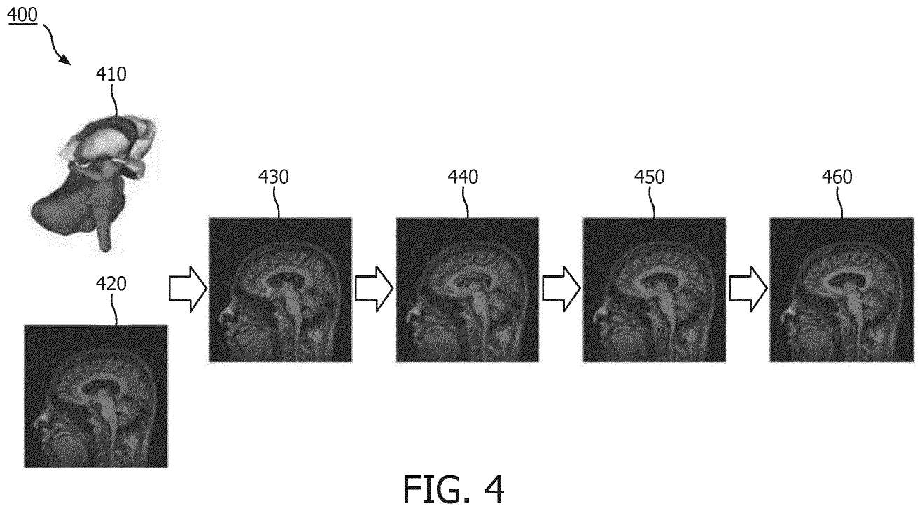

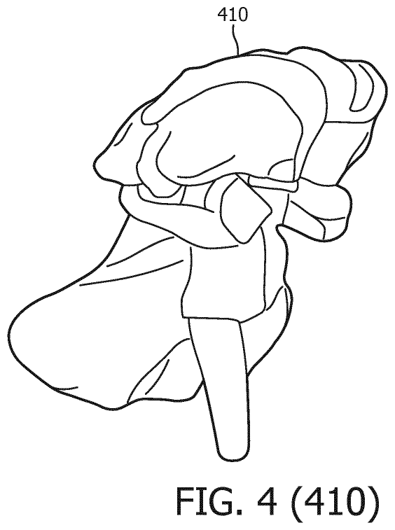

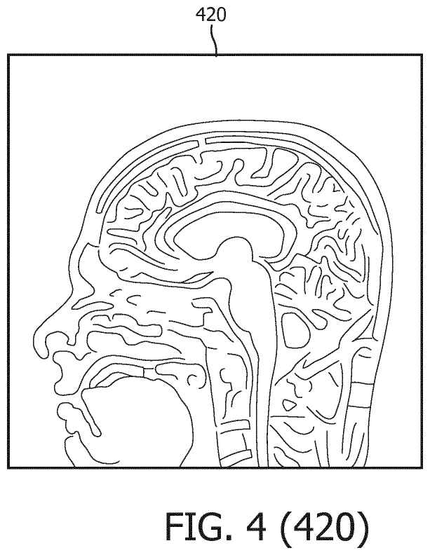

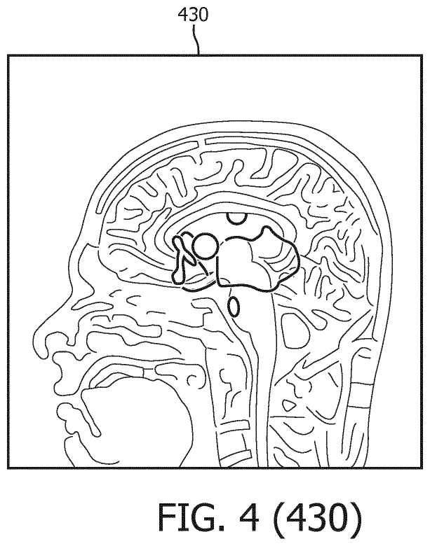

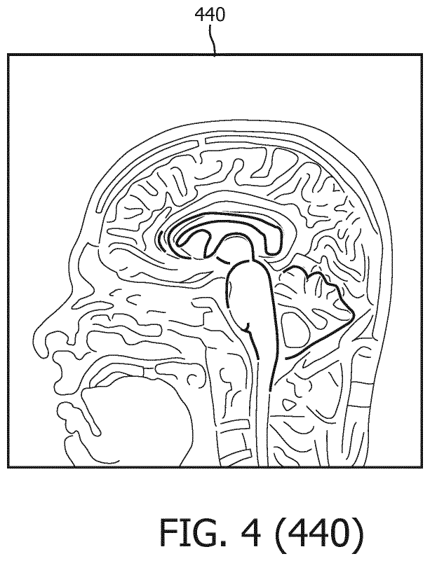

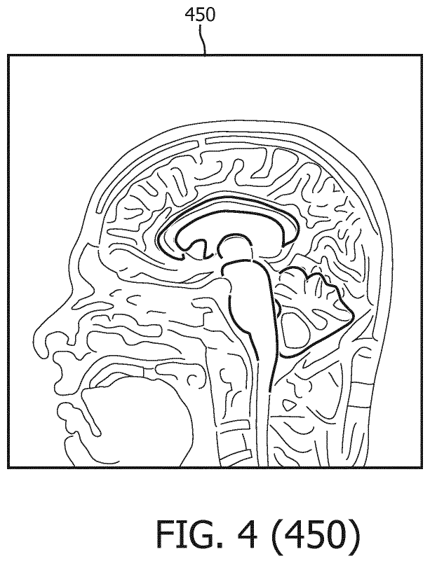

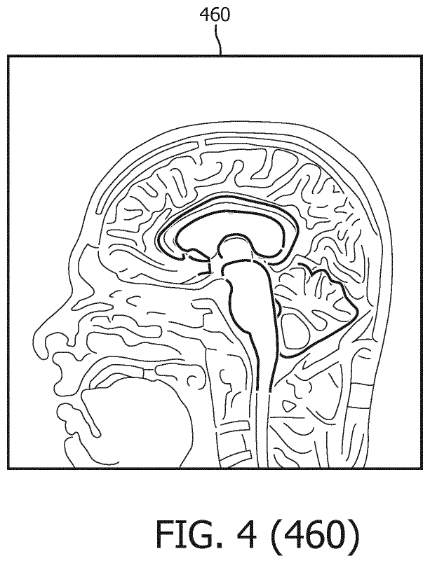

At operation 202 of the method 200, MR data of the subject's brain is obtained by use of the MRI device 110 in communication with the computing device 120. The computing device 120 can process the MR data of a subject's brain and output MR image data to the display 160 to display an MR image, such as the MR image 420 in FIG. 4. In some embodiments, the MR data includes T1W MR data. While the MR image 420 shown in FIG. 4 is a top view of the subject's brain, a person of ordinary skill in the art would understand that MR images of the subject's brain viewed from other directions can be obtained or derived by the computing device 120 as well. The MR data obtained at operation 202 includes MR data of anatomical structures in the subject's brain.

At operation 204 of the method 200, the MR data of the subject's brain are segmented to delineate a first geometry of a first anatomical structure and a second geometry of a second anatomical structure in the subject's brain.

Referring now to FIG. 4, shown therein is a process flow 400 for segmenting the MR data to delineate geometries of anatomical structures in the brain of the subject. FIG. 4 was originally prepared as a color drawing because representing various aspects in black and white on a black and white medical image is challenging. At the time of filing the present application, most patent offices around the world do not accept color drawings. Therefore, to help illustrate the aspects shown in FIG. 4, additional figures FIG. 4 (410), FIG. 4 (420), FIG. 4 (430), FIG. 4 (440), FIG. 4 (450), and FIG. 4 (460) are provide to illustrate in black and white various aspects previously shown in color in FIG. 4. Any discrepancies between FIG. 4 and any of FIG. 4 (410), FIG. 4 (420), FIG. 4 (430), FIG. 4 (440), FIG. 4 (450), and FIG. 4 (460) should be construed in favor of FIG. 4, which is the original. The color version of FIG. 4 may be available from the U.S. Patent and Trademark Office in US patent applications related to the present patent application.

In some embodiments, the computing device 120 can segment the MR data of the subject's brain, represented by the MR image 420, based on a 3D brain model 410. In some embodiments, the 3D brain model 410 can be a shape-constrained deformable brain model. In some instances, the 3D brain model 410 may be the brain model described in “Evaluation of traumatic brain injury subjects using a shape-constrained deformable model,” by L. Zagorchev, C. Meyer, T. Stehle, R. Kneser, S. Young and J. Weese, 2011, in Multimodal Brain Image Analysis by Liu. T., Shen D., Ibanez L., Tao X. (eds). MBIA 2011. Lecture Notes in Computer Science, vol. 7012. Springer, Berlin, Heidelberg, the entirety of which is hereby incorporated by reference. In some instances, the 3D brain model may be the deformable brain model described in U.S. Pat. No. 9,256,951, titled “SYSTEM FOR RAPID AND ACCURATE QUANTITATIVE ASSESSMENT OF TRAUMATIC BRAIN INJURY” or the shape-constrained deformable brain model described in U.S. Pat. App. Pub. No. 20150146951, titled “METHOD AND SYSTEM FOR QUANTITATIVE EVALUATION OF IMAGE SEGMENTATION,” each of which is hereby incorporated by reference in its entirety. In some implementations, the 3D brain model 410 is stored in the computing device 120 or a storage device or medium retrievable by the computing device 120. Operation 204 can be performed simultaneously with or subsequently after operation 202.

As shown in MR image 430, the 3D brain model 410 is initialized by being matched to the MR image 420 of the brain. Then a generalized Hough transformation (GHT) is performed on the 3D brain model 410 to match the 3D brain model 410 to the geometries of the anatomical structures in the MR image 420 in terms of location and orientation, as illustrated in MR image 440. Thereafter, as shown in MR image 450, the 3D brain model 410 goes through parametric adaptation where location, orientation and scaling are adjusted using a global similarity transformation and/or a multi-linear transformation to better adapt to the anatomical structures in the MR image 420. As illustrated by MR image 460, the 3D brain model 410 undergoes deformable adaptation where multiple iterations of boundary detection and adjustment of meshes in 3D brain model 410 are performed to adapt the 3D brain model to anatomical structures in the brain.

At operation 206 of the method 200, fMRI data of the subject's brain is obtained. fMRI relies on the fact the oxygenated hemoglobin and deoxygenated hemoglobin has different magnetic properties that result in different magnetic resonance (MR) signal intensities. Because the cerebral blood flow bears a direct correlation with neuronal activation, by measuring the blood demand in a brain region, fMRI measures activation levels of that brain region. In addition, because the demand for blood can represent demand for oxygen, fMRI can also be a tool and technique to measure oxygen demand in a brain region. During an fMRI scan, a task designed to increase regional brain activities is administered to a subject and the MRI device can detect changes in the ratio of oxygenated and deoxygenated blood. Operation 206 can be performed simultaneously with or subsequently after operations 202 and 204.

For example, the task can be a dual N-back task. In a dual N-back task, a subject is presented with a series of visual stimuli and auditory stimuli simultaneously. In some implementations, a subject starts with a 1-back condition, where he/she is required to provide an affirmative response if the present visual stimulus matches the immediately preceding visual stimulus. Likewise, if the present auditory stimulus matches the immediately preceding auditory stimulus, the subject is required to provide an affirmative response. If both the present visual and auditory stimuli match the immediately preceding visual and auditory stimuli, the subject is asked to provide a double affirmative response. If none of the stimuli matches, no response is required. If the accuracy rate of the subject reaches a certain level, the n-back level is increased by one (e.g. from 1-back to 2-back). If the accuracy level falls below a certain level, the n-back level is decrease by one (e.g. from 3-back to 2-back). In some instances, if the accuracy level of the subject is maintained at a certain level, the n-back level remains unchanged. The dual N-back task is described in Susanne M. Jaeggi et al., Improving Fluid Intelligence with Training on Working Memory, Pro. Natl. Acad. Sc. U.S. A., 2008 May 13; 105(19): 6829-6833. FIG. 4 shows activation levels in control subjects' brains and activation levels in brains of subjects with mild traumatic brain injury (MTBI) when the subjects were subjected to dual N-back tasks. Without anatomical-structure-specific activation levels, the activation levels within a specific anatomical structure cannot be quantified and meaningfully associated to a specific neurological disorder. The systems and methods of the present disclosure achieve just that. By segmenting the MR data and aligning the MR data with the fMRI data, the activation level in each of the geometries of the anatomical structures can be determined. The activation level used herein can be an accumulated activation level, an instantaneous activation level, a time-average activation level, or an event-average activation level.

The operation 206 can be demonstrated in conjunction with FIG. 5, which shows an MR image 500 of a brain of a subject overlaid with highlighted boundaries of the geometries of anatomical structures, including a geometry of thalamus 510 and corpus callosum 520. In some embodiments, the computing device 120 can determine a first activation level within a first geometry (for example, the geometry of the thalamus 510) and a second activation level within a second geometry (for example, the geometry of the corpus callosum 520). As shown in FIG. 5, the first activation level can be represented by a first graphical overlay 610 and the second activation level can be represented by a second graphical overlay 620. The first and second activation levels here can be accumulated activation levels, instantaneous activation levels, time-average activation levels, or event-average activation levels. In addition, the computing device 120 can determine a pattern or sequence of the activation in different anatomical structures. For example, the first activation level in the geometry of thalamus 510 may increase while the second activation level in the geometry of the corpus callosum 520 is on the increase and then the second activation level can increase in response to a dual N-back task while the first activation level wanes in response to the same task. Besides quantitative intensities of activation levels, the pattern/sequence of the activation among different anatomical structure in response to a task or stimulation can also be indicative of a neurological disorder or condition.

At operation 208 of the method 200, EEG data of the brain of the subject is obtained. By segmenting the MR data and aligning the MR data with the EEG data, the electrical activity level within each of the geometries of the anatomical structures can be determined. Operation 208 can be performed simultaneously with or subsequently after operations 202, 204, and 206.

At operation 210 of the method 200, MEG data of the brain of the subject is obtained. By segmenting the MR data and aligning the MR data with the MEG data, the neuronal activity level within each of the geometries of the anatomical structures can be determined.

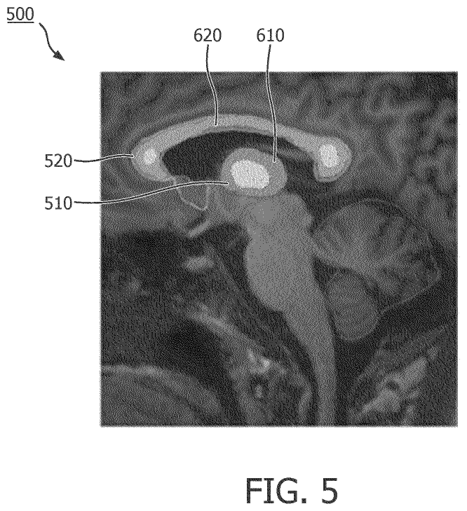

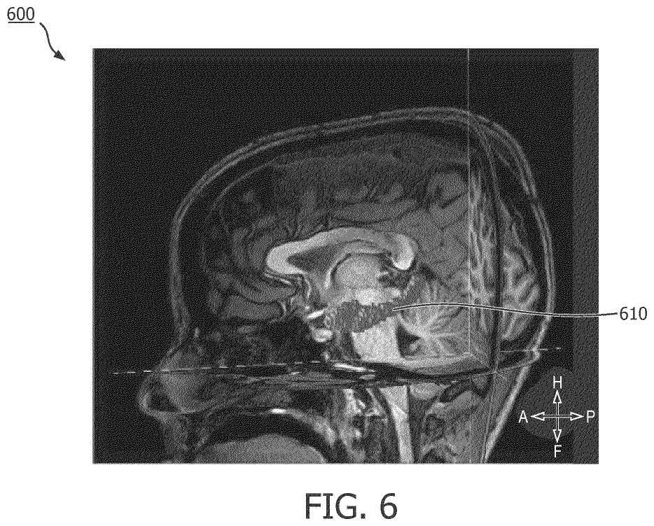

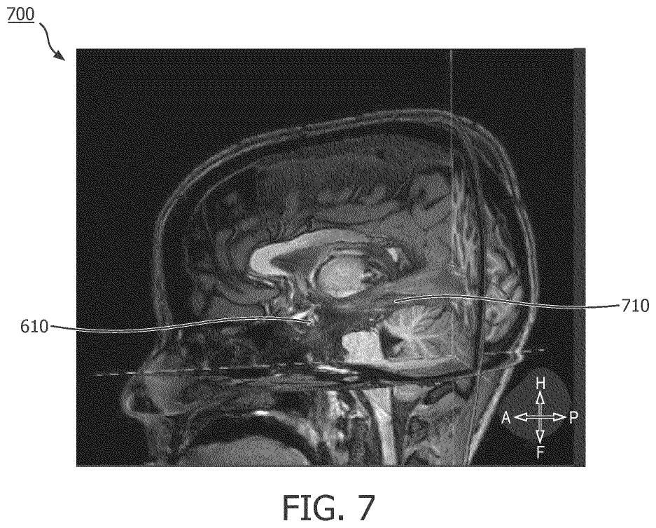

At operation 212 of the method 200, DTI data of the brain of the subject is obtained. By segmenting the MR data and aligning the MR data with the MEG data, the computing device 120 can identify fiber tracts that go through the anatomical structure and determine the fiber tract density or a ratio of fiber tract volume within the anatomical structure. Operation 212 can be demonstrated in conjunction with FIGS. 6 and 7. One of the ways the computing device 120 segments an anatomical structure is by representing the anatomical structure in voxels. An exemplary voxel representation is demonstrated by FIG. 6, where an MR image 600 of the subject includes a segmented representation 610 of the subject's amygdala-hippocampal complex (AHC). The segmented representation 610 includes voxels that fill the geometry of the subject's AHC. While FIG. 6 shows the segmented representation 610 of the subject's AHC, people of ordinary skill in the art would understand that such segmentation can be done to all brain anatomical structures. Referring now to FIG. 7, shown therein is an MR image 700 of the subject's brain overlaid with fiber tracts 710 passing through the segmented representation 610 of the subject's AHC. In some embodiments, the voxels in the segmented representation 610 can serve as the starting point or “seed” to track the fiber tracts 710 passing through them, allowing the fiber tracts 710 to be identified at operation 212. Operation 212 can be performed simultaneously with or subsequently after operations 202, 204, 206, and 208.

At operation 214 of the method 200, the activation levels, the sequence of activation, the electrical activity levels, the neuronal activity levels, the fiber tract densities are associated with a diagnosis of the subject's brain. Put in context of the system 100 shown in FIG. 1, at operation 214, the computing device 120 receives a diagnosis of the patent with respect to the brain. If the diagnosis is negative and indicative of a healthy brain, the computing device 120 then associates the fMRI activation levels, sequence of activation, DTI fiber tract densities, MEG neuronal activity levels, EEG electrical activity levels within respective anatomical structures of the brain with a negative diagnosis or a neurologically non-diseased subject. If, however, the diagnosis is positive and indicative of a neurological disorder, then the computing device 120 associates the aforementioned anatomical structure specific data with a neurologically diseased subject. In some embodiments, whenever the diagnosis is positive, any information on a treatment recommendation for recommended therapies or procedures, and a prescription recommendation, for recommended medication, are also associated with the subject diagnosed with a neurological disorder.

The method 200 then bifurcates into operation 216A and operation 216B. At operation 216A, the fMRI activation levels, sequence of activation, DTI fiber tract densities, MEG neuronal activity levels, EEG electrical activity levels within respective anatomical structures associated with a negative diagnosis or a neurologically non-diseased subject are stored in a normative database, such as the normative database 170 shown in FIG. 1. Over time, the normative database can include fMRI activation levels, sequence of activation, DTI fiber tract densities, MEG neuronal activity levels, EEG electrical activity levels within respective anatomical structures of a plurality of neurologically non-diseased subjects. At operation 216B, the fMRI activation levels, sequence of activation, DTI fiber tract densities, MEG neuronal activity levels, EEG electrical activity levels within respective anatomical structures associated with a neurologically diseased subject are stored in a biomarker database, such as the biomarker database 180 shown in FIG. 1. Over time, the biomarker database can include fMRI activation levels, sequence of activation, DTI fiber tract densities, MEG neuronal activity levels, EEG electrical activity levels within respective anatomical structures of a plurality of neurologically diseased subjects. In instances where a treatment recommendation and/or a prescription recommendation are associated with a neurologically diseased subject diagnosed with the neurological disorder, the treatment recommendation and the prescription recommendation are also stored in the biomarker database 180. For example, subjects diagnosed with epilepsy are put on antiepileptic drugs. Their treatments and/or prescription recommendations can be stored in the database along with their associated imaging (such as fMRI, EEG, MEG, and DTI), and/or non-imaging data (such as genomics, clinical essays, electronic medical records, radiology reports), and/or prior treatment recommendations. In some embodiments, the data in normative database and the biomarker database can be sorted based on age, gender, race, or combinations thereof.

Referring now to FIG. 3, shown therein is a method 300 for determining an abnormality in an anatomical structure in a brain of a subject and a probability of a neurological disorder. The method 300 includes operations 302, 304, 306, 308, 310, 312, 314, 316, and 318. It is understood that the operations of method 300 may be performed in a different order than shown in FIG. 3, additional operations can be provided before, during, and after the operations, and/or some of the operations described can be replaced or eliminated in other embodiments. The operations of the method 300 can be carried out by a computing device in the MRI system, such as the computing device 120 of the system 100. The method 300 will be described below with reference to FIGS. 3, 4, 5, 6, and 7. As operations 302, 304, 306, 308, 310, and 312 of the method 300 bear resemblance to operations 202, 204, 206, 208, 210, and 212 of method 200, they will be described in less detail below.

At operation 302 of the method 300, MR data of the subject's brain is obtained by use of the MRI device 110 in communication with the computing device 120.

At operation 304 of the method 200, the MR data of the subject's brain are segmented to delineate a first geometry of a first anatomical structure and a second geometry of a second anatomical structure in the subject's brain.

At operation 306 of the method 300, fMRI data of the subject's brain is obtained. The fMRI data are aligned with the MR data either through survey scans or through suitable alignment processes, such volume localization and direction cosines. By segmenting the MR data and aligning the MR data with the fMRI data, a test activation level in each of the geometries of the anatomical structures can be determined. The test activation level used herein can be an accumulated activation level, an instantaneous activation level, a time-average activation level, or an event-average activation level.

At operation 308 of the method 300, EEG data of the brain of the subject is obtained. The EEG data are aligned with the MR data through suitable alignment processes, such as survey scans, rigid registration, volume localization and direction cosines. By segmenting the MR data and aligning the MR data with the EEG data, a test electrical activity level within each of the geometries of the anatomical structures can be determined.

At operation 310 of the method 300, MEG data of the brain of the subject is obtained. The MEG data are aligned with the MR data through suitable alignment processes, such as survey scans, rigid registration, volume localization and direction cosines. By segmenting the MR data and aligning the MR data with the MEG data, a test neuronal activity level within each of the geometries of the anatomical structures can be determined.

At operation 312 of the method 300, DTI data of the brain of the subject is obtained. The DTI data are aligned with the MR data either through survey scans or through suitable alignment processes, such as survey scans, rigid registration, volume localization and direction cosines. By segmenting the MR data and aligning the MR data with the MEG data, the computing device 120 can identify fiber tracts that go through the anatomical structure and determine the fiber tract density within the anatomical structure.

At operation 314 of the method 300, an abnormality in the anatomical structure is determined the computing device 120 by comparing the test activation level, the test electrical activity level, the test neuronal activity level, and the test fiber tract density with data in a normative database, such as the normative database 170. As described above with respect to operation 216A of the method 200, the normative database can include fMRI activation levels, sequence of activation, DTI fiber tract densities, MEG neuronal activity levels, EEG electrical activity levels within respective anatomical structures of a plurality of neurologically non-diseased subjects. In some embodiments, the data in the normative database are normalized based on the subject's age, gender, sex, and/or head size before they are compared to the subject's test activation level, the test electrical activity level, the test neuronal activity level, and the test fiber tract density. In some embodiments, an abnormality in an anatomical structure is determined if the subject's test activation level, the test electrical activity level, the test neuronal activity level, and the test fiber tract density within the anatomical structure deviates from normative values by a threshold percentage. In some implementations, the threshold percentage is a percentage determined based on a cross comparison between the data in the normative database to a biomarker database. In some implementations, the threshold percentage is a fraction of the standard deviation of the normalized data.

At operation 316 of the method 300, a probability of neurological disorder is determined by comparing the test activation level, the test electrical activity level, the test neuronal activity level, and the test fiber tract density associated with the abnormality to data in the biomarker database. As described above with respect to operation 216B of the method 200, the biomarker database can include fMRI activation levels, sequence of activation, DTI fiber tract densities, MEG neuronal activity levels, EEG electrical activity levels within respective anatomical structures of a plurality of neurologically diseased subjects. In some embodiments, the data in the biomarker database are normalized based on the subject's age, gender, sex, and/or head size before they are compared to the subject's test activation level, the test electrical activity level, the test neuronal activity level, and the test fiber tract density. In some embodiments, a probability of a neurological disorder is determined by matching the subject's test activation level, the test electrical activity level, the test neuronal activity level, and the test fiber tract density within the anatomical structure to biomarkers of the neurological disorder in the biomarker database. For example, if the data in the biomarker database statistically indicate that a neurologically diseased subject with an X activation level and Y neuronal activity level in anatomical structure Z has a 95% probability of being diagnosed with a neurological disorder A and the subject's test data match or exceed X activation level and Y neuronal activity level in anatomical structure Z, then the probability of the neurological disorder A for the subject is 95%. In some implementations, the data in the biomarker database can be cross compared to the data in the normative database to generate a threshold percentage for determining an abnormality in operation 314. For example, if an average activation level within an anatomical structure in the biomarker database is 15% higher than it counterpart in the normative database, then 15% can serve as the threshold percentage for the purposes of determining an abnormality within the anatomical structure in operation 314.

In some embodiments, operations 314 and 316 are performed in parallel and independently from each other. In those embodiments, the computing device 120 accesses both databases simultaneously and performs the comparisons required in operations 314 and 316 separately. In some other embodiments, operations 314 and 316 are performed in sequence and operation 316 depends on result of operation 314. In those embodiments, one an abnormality is identified with respect to an anatomical structure, operation 316 is performed only with respect to that “abnormal” anatomical structure to generate a convergent result.

At operation 318 of the method 300, a graphical representation of the abnormality, the probability of the neurological disorder are output to a display, such as the display 160. As described above, the biomarker database can include information on a treatment recommendation for recommended therapy or procedures, and a prescription recommendation, for recommended medication. In those embodiments, the graphical representation can also include the treatment recommendation and the prescription recommendation for a neurological disorder if the probability of the neurological disorder is greater than 0%. In some other embodiments, the graphical representation only includes the treatment recommendation and the prescription recommendation for a neurological disorder if the probability of the neurological disorder is greater than 50%. In some implementations, the graphical representation can include color contours, text, pop-up dialog boxes, clickable hyperlinks. In some implementation, the graphical representation can assume a form of a radiology report.

The systems, devices, and methods of the present disclosure can include features described in U.S. Provisional App. Ser. No. ______ (Atty. Dkt. No. 2017PF02586/44755.1862 PV01), the entireties of which is hereby incorporated by reference herein.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. A system for evaluating an anatomical structure in a brain of a subject, comprising:
a computing device in communication with a magnetic resonance imaging (MRI) device, the computing device operable to:
determine an abnormality in the anatomical structure by comparing a test activation level within a geometry of the anatomical structure to data in a normative database, the test activation level being determined by aligning functional magnetic resonance imaging (fMRI) data obtained by use of the MRI device and the geometry of the anatomical structure, the geometry of the anatomical structure being delineated based on segmentation of magnetic resonance (MR) data obtained by use of the MRI device, wherein the data in the normative database include activation levels of the anatomical structure of a plurality of neurologically non-diseased subjects; and
output, to a display device, a graphical representation of the abnormality in the anatomical structure.

2. The system of claim 1, wherein the computing device is further operable to:
determine a probability of a neurological disorder by comparing the test activation level associated with the abnormality to data in a biomarker database,
wherein the data in the biomarker database include activation levels of the anatomical structure of a plurality of neurologically diseased subjects, and
wherein the graphical representation includes the probability of the neurological disorder.

3. The system of claim 2, wherein the computing device is further operable to:
determine the abnormality in the anatomical structure by comparing a test electrical activity level within the geometry of the anatomical structure to the data in the normative database, the test electrical activity level being determined by aligning electroencephalography (EEG) data obtained by use of an EEG device and the geometry of the anatomical structure; and
determine the probability of the neurological disorder by comparing the test electrical activity level associated with the abnormality to the data in the biomarker database,
wherein the computing device is in communication with the EEG device,
wherein the data in the normative database include electrical activity levels of the anatomical structure of the plurality of neurologically non-diseased subjects, and
wherein the data in the biomarker database include electrical activity levels of the anatomical structure of the plurality of neurologically diseased subjects.

4. The system of claim 2, wherein the computing device is further operable to:
determine the abnormality in the anatomical structure by comparing a test neuronal activity level within the geometry of the anatomical structure to the data in the normative database, the test neuronal activity level being determined by aligning magnetoencephalography (MEG) data obtained by use of an MEG device and the geometry of the anatomical structure; and
determine the probability of the neurological disorder by comparing the test neuronal activity level associated with the abnormality to the data in the biomarker database,
wherein the computing device is in communication with the MEG device,
wherein the data in the normative database include neuronal activity levels of the anatomical structure of the plurality of neurologically non-diseased subjects, and
wherein the data in the biomarker database include neuronal activity levels of the anatomical structure of the plurality of neurologically diseased subjects.

5. The system of claim 2, wherein the computing device is further operable to:
determine the abnormality in the anatomical structure by comparing a test fiber tract density within the geometry of the anatomical structure to the data in the normative database, the test fiber tract density being determined by aligning diffusion tensor imaging (DTI) data obtained by use of the MRI device and the geometry of the anatomical structure; and
determine the probability of the neurological disorder by comparing the test fiber tract density associated with the abnormality to the data in the biomarker database,
wherein the data in the normative database include fiber tract densities of the anatomical structure of the plurality of neurologically non-diseased subjects, and
wherein the data in the biomarker database include fiber tract densities of the anatomical structure of the plurality of neurologically diseased subjects.

6. The system of claim 2, wherein the graphical representation includes a treatment recommendation.

7. The system of claim 2, wherein the graphical representation includes a prescription recommendation.

8. The system of claim 2, wherein the graphical representation comprises a report.

9. The system of claim 2, further comprising the MRI device and the display device.

10. A system for evaluating an anatomical structure in a brain of a subject, comprising:
a computing device in communication with a magnetic resonance imaging (MRI) device, the computing device operable to:
determining a probability of a neurological disorder associated with an abnormality in the anatomical structure by comparing a test activation level within a geometry of the anatomical structure to data in a biomarker database, the test activation level being determined by aligning functional magnetic resonance imaging (fMRI) data obtained by use of the MRI device and the geometry of the anatomical structure, the geometry of the anatomical structure being delineated based on segmentation of magnetic resonance (MR) data obtained by use of the MRI device, wherein the data in the biomarker database include activation levels of the anatomical structure of a plurality of neurologically diseased subjects; and
output, to a display device, a graphical representation of the probability of the neurological disorder.

11. The system of claim 10, wherein the computing device is further operable to:
determine an abnormality in the anatomical structure by comparing a test activation level within the geometry of the anatomical structure to data in a normative database,
wherein the data in the normative database include activation levels of the anatomical structure of a plurality of neurologically non-diseased subjects, and
wherein the graphical representation includes the abnormality in the anatomical structure.

12. The system of claim 11, wherein the computing device is further operable to:
determine the abnormality in the anatomical structure by comparing a test electrical activity level within the geometry of the anatomical structure to the data in the normative database, the test electrical activity level being determined by aligning electroencephalography (EEG) data obtained by use of an EEG device and the geometry of the anatomical structure; and
determine the probability of the neurological disorder by comparing the test electrical activity level to the data in the biomarker database,
wherein the computing device is in communication with the EEG device,
wherein the data in the normative database include electrical activity levels of the anatomical structure of the plurality of neurologically non-diseased subjects, and
wherein the data in the biomarker database include electrical activity levels of the anatomical structure of the plurality of neurologically diseased subjects.

13. The system of claim 11, wherein the computing device is further operable to:
determine the abnormality in the anatomical structure by comparing a test neuronal activity level within the geometry of the anatomical structure to the data in the normative database, the test neuronal activity level being determined by aligning magnetoencephalography (MEG) data obtained by use of an MEG device and the geometry of the anatomical structure; and
determine the probability of the neurological disorder by comparing the test neuronal activity level to the data in the biomarker database,
wherein the computing device is in communication with the MEG device,
wherein the data in the normative database include neuronal activity levels of the anatomical structure of the plurality of neurologically non-diseased subjects, and
wherein the data in the biomarker database include neuronal activity levels of the anatomical structure of the plurality of neurologically diseased subjects.

14. The system of claim 11, wherein the computing device is further operable to:
determine the abnormality in the anatomical structure by comparing a test fiber tract density within the geometry of the anatomical structure to the data in the normative database, the test fiber tract density being determined by aligning diffusion tensor imaging (DTI) data obtained by use of the MRI device and the geometry of the anatomical structure; and
determine the probability of the neurological disorder by comparing the test fiber tract density to the data in the biomarker database,
wherein the data in the normative database include fiber tract densities of the anatomical structure of the plurality of neurologically non-diseased subjects, and
wherein the data in the biomarker database include fiber tract densities of the anatomical structure of the plurality of neurologically diseased subjects.

15. The system of claim 10, wherein the graphical representation includes a treatment recommendation.

16. The system of claim 10, wherein the graphical representation includes a prescription recommendation.

17. The system of claim 10, wherein the graphical representation comprises a report.

18. The system of claim 10, further comprising the MRI device and the display device.
